(19) United States
(12) Patent Application Publication
(10) Pub. No.: US 2024/0293668 A1
(43) Pub. Date: Sep. 5, 2024

(54) MONOLITHIC PERICARDIAL PACEMAKERS

(71) Applicants: CHILDREN’S HOSPITAL LOS ANGELES, Los Angeles, CA (US); UNIVERSITY OF SOUTHERN CALIFORNIA, Los Angeles, CA (US)

(72) Inventors: Yaniv Bar-Cohen, South Pasadena, CA (US); Gerald E. Loeb, South Pasadena, CA (US); Raymond A. Peck, Los Angeles, CA (US); Samuel Kohan, Fountain Valley, CA (US); Tristan Lerner, Torrance, CA (US); Allison Hill, Los Angeles, CA (US); Jay D. Pruetz, Culver City, CA (US); Mark H. Shwayder, Hermosa Beach, CA (US); Michael J. Silka, La Canada, CA (US)

(21) Appl. No.: 18/573,421

(22) PCT Filed: Jun. 23, 2022
(86) PCT No.: PCT/US2022/034802

§ 371 Date: Dec. 21, 2023

Related U.S. Application Data
(60) Provisional application No. 63/214,170, filed on Jun. 23, 2021.

(51) Int. Cl. A61N 1/05

(52) U.S. Cl. CPC A61N 1/0592; A61N 1/0568; A61N 1/059

(57) ABSTRACT

Pacemakers and pacemaker delivery systems and methods are disclosed which use a modified cylindrical pacemaker body having a flat side which includes the anode/cathode and spikes to anchor the pacemaker in place within the pericardium.

This Patent Application claims priority to U.S. Provisional Patent Application Ser. No. 63/214,170, filed Jun. 23, 2021, the content of which is hereby incorporated by reference herein in its entirety into this disclosure.

### TECHNICAL FIELD

The present subject disclosure relates generally to cardiac pacemakers. More specifically, the present subject disclosure relates to monolithic pericardial pacemakers.

### BACKGROUND OF THE SUBJECT DISCLOSURE

The heart is a hollow muscular structure that resides inside a baglike membrane called the pericardium. There are many medical conditions in which the heart does not beat at a reliable rate or at a sufficiently high rate to sustain healthy life. Since their introduction in the 1950s, cardiac pacemakers have generally taken the form of a surgically implanted electrical pulse generator powered by a primary cell and connected to one or more flexible, endovascular leads that pass through the major veins into the interior of the right atrium and/or right ventricle, where they are anchored to the myocardial muscle. One of the most common forms of failure is stress fatigue of the leads from constant bending with each heartbeat.

For patients who are too small to accommodate such endovascular leads, have cardiac anatomic abnormalities that preclude transvenous lead placement or have infections or blood clotting or other disorders that contraindicate such endovascular leads, epicardial leads may be sutured to the outer surface of the myocardium. This requires major surgery to open the chest and pericardium to gain access to the epicardial surface.

It is generally desirable to eliminate the need for open surgical implantation of any medical device and to eliminate the need for repeat implantation procedures when the primary power source is depleted. Any open surgery entails risks from anesthesia and wound-healing and generally results in substantial pain, discomfort, and limited motor function for days to weeks after surgery. Making the implant small enough to enable minimally invasive implantation techniques necessarily compromises the amount of energy that can be stored in the implant. It is now possible to use one or more rechargeable cells to power the implant and to recharge those cells from outside the body by inductive or other transcutaneous transmission of electrical power. Nevertheless, it is important to conserve electrical energy by minimizing the strength of the electrical pulses required to capture the heart rate so to minimize battery drain.

More recently. “leadless pacemakers” have been developed which are intended to be intravenously attached to the endocardial wall of the right ventricle as a single, free-standing module. Nevertheless, such leadless pacemakers still result in inflammation, fibrosis, and can get dislodged from their positions by exposure to repeated cardiac motion. Such events tend to increase the strength of the electrical pulses required to capture the heart rate, thereby shortening the life of the power supply.

### SUMMARY OF THE SUBJECT DISCLOSURE

In the present subject disclosure, Applicants have developed a pacemaker with a unique shape and configuration which is low profile, can be implanted using minimally invasive techniques, and is structurally designed to fit within the pericardium while minimizing inflammation and the formation of scar tissue. Further, the pacemaker's unique shape and structure prevent or minimize rolling or translational movement by fitting within the pericardium, which secures it therein.

In one exemplary embodiment, the present subject disclosure is a pacemaker device. The pacemaker includes an elongated, modified cylindrical shell having a leading end and a trailing end, and an elongated flattened side which extends from the leading end to the trailing end; wherein in a cross section of the round modified cylindrical shell, the flattened side is a chord across the cross section. The pacemaker has a flat electrode surface positioned on the flattened side that functions as a cathode to the pacemaker device.

In another exemplary embodiment, the present subject disclosure is a pacemaker delivery assembly. The assembly includes an elongated cannula; an insertion tool positionable within the cannula and having a proximal end and a distal end; and a pacemaker positionable within the cannula, adapted to engage with the insertion tool, and comprising: an elongated, modified cylindrical shell having a leading end and a trailing end, and an elongated flattened side which extends from the leading end to the trailing end; wherein in a cross section of the round modified cylindrical shell, the flattened side is a chord across the cross section.

In another exemplary embodiment, the present subject disclosure is a method of inserting a pacemaker. The method includes threading a string around a pulley on one end of the pacemaker; mounting the pacemaker on a distal end of an insertion tool; reversibly attaching both ends of the string to a proximal end of the insertion tool; and inserting the insertion tool and pacemaker within a cannula to deliver to a target location on an epicardial surface.

### BRIEF DESCRIPTION OF THE DRAWINGS

The accompanying drawings, which are incorporated in and constitute a part of this specification, illustrate embodiments of the subject disclosure and technical data supporting those embodiments, and together with the written description, serve to explain certain principles of the subject disclosure.

FIG. 1A shows an oblique view of a pacemaker delivery assembly, according to an exemplary embodiment of the present subject disclosure.

FIG. 1B shows a detailed view of a pacemaker delivery assembly disposed within a cannula, according to an exemplary embodiment of the present subject disclosure.

FIG. 1C shows a detailed view of a pacemaker delivery assembly as the pacemaker is being released from the insertion tool, according to an exemplary embodiment of the present subject disclosure.

FIG. 2A shows a top oblique view of a pacemaker, according to an exemplary embodiment of the present subject disclosure.

FIG. 2B shows a bottom oblique view of the pacemaker of FIG. 2A, according to an exemplary embodiment of the present subject disclosure.

FIG. 3A shows a detailed side view of a pacemaker delivery assembly before release of the pacemaker, according to an exemplary embodiment of the present subject disclosure.

FIG. 3B shows a detailed side view of a pacemaker delivery assembly during release of the pacemaker, according to an exemplary embodiment of the present subject disclosure.

FIG. 4A shows a side view of a pacemaker assembly, according to another exemplary embodiment of the present subject disclosure.

FIG. 4B shows a back view of a pacemaker assembly, according to another exemplary embodiment of the present subject disclosure.

FIG. 4C shows a front view of a pacemaker assembly, according to another exemplary embodiment of the present subject disclosure.

FIG. 5A shows a side view of a pacemaker, according to another exemplary embodiment of the present subject disclosure.

FIG. 5B shows a cross-sectional view along plane A-A of the pacemaker of FIG. 5A, according to another exemplary embodiment of the present subject disclosure.

FIG. 5C shows a bottom view of a pacemaker, according to another exemplary embodiment of the present subject disclosure.

FIG. 5D shows a back view of a pacemaker, according to another exemplary embodiment of the present subject disclosure.

FIG. 6A shows a bottom view of a pacemaker, according to another exemplary embodiment of the present subject disclosure.

FIG. 6B shows a cross-sectional view along plane B-B of the pacemaker of FIG. 6A, according to another exemplary embodiment of the present subject disclosure.

FIG. 7A shows a perspective view of a pushrod, according to another exemplary embodiment of the present subject disclosure.

FIG. 7B shows a side view of a pushrod, according to another exemplary embodiment of the present subject disclosure.

FIG. 7C shows a front view of a pushrod, according to another exemplary embodiment of the present subject disclosure.

FIG. 7D shows a cross-sectional view along plane C-C of the pushrod of FIG. 7C, according to another exemplary embodiment of the present subject disclosure.

FIG. 8 shows a perspective view of a pacemaker assembly, according to yet another exemplary embodiment of the present subject disclosure.

FIG. 9 shows side, bottom, and end views of a pacemaker assembly, according to yet another exemplary embodiment of the present subject disclosure.

### DETAILED DESCRIPTION OF THE SUBJECT DISCLOSURE

The following detailed description references specific embodiments of the subject disclosure and accompanying figures, including the respective best modes for carrying out each embodiment. It shall be understood that these illustrations are by way of example and not by way of limitation.

The present subject disclosure describes highly effective and simple to use devices, assemblies, and methods which comprise a monolithic pacemaker implanted through a percutaneous pericardial approach and placed at the epicardial surface without pacing leads. The pacemaker will remain in place and have minimal inflammation at the electrode. Repositioning/removal is possible using the devices and techniques presented herein.

The pacemaker described herein has a unique shape in that it is substantially a cylinder except for a flattened side. The electrode is centered at that flattened side, and there are both downward projecting spikes and a textured/serrated surface to prevent sliding. The device may be deployed from a shelf on a specially designed insertion tool.

The device is also leadless without being on the inside of the heart, eliminating the risk of embolization. It is possible to place multiple devices either initially or at different times. Devices may be implanted on any part of the heart and may coordinate their monitoring and pacing functions as is well-known in the art. With this device, it is possible to pace the left ventricle directly. Its insertion may be accomplished by a minimally invasive procedure, as described herein. The device may also be implanted onto the epicardial surface of the heart using conventional open-chest surgery and it may be affixed to the surface using conventional means such as sutures as is well-known in the art.

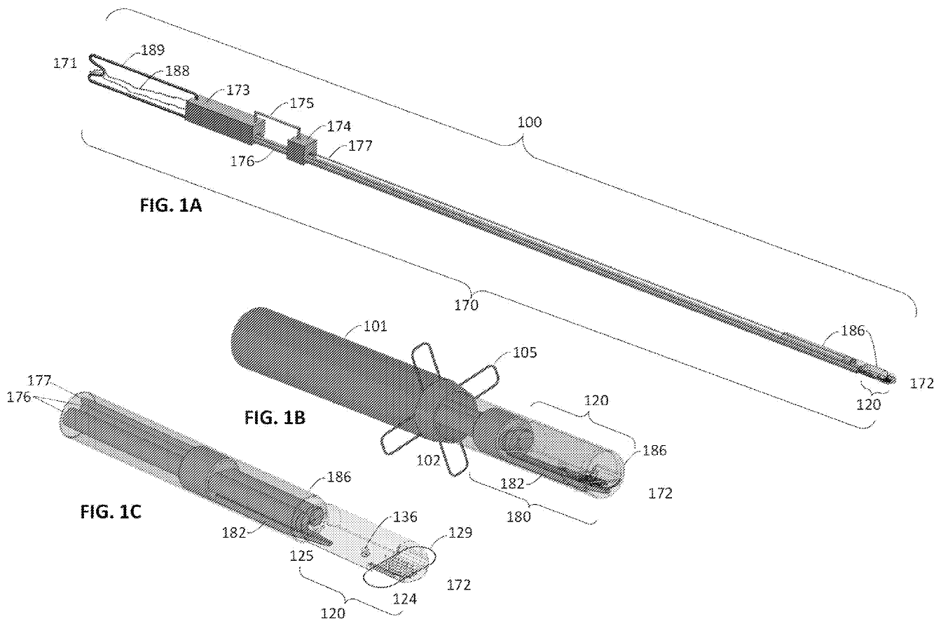

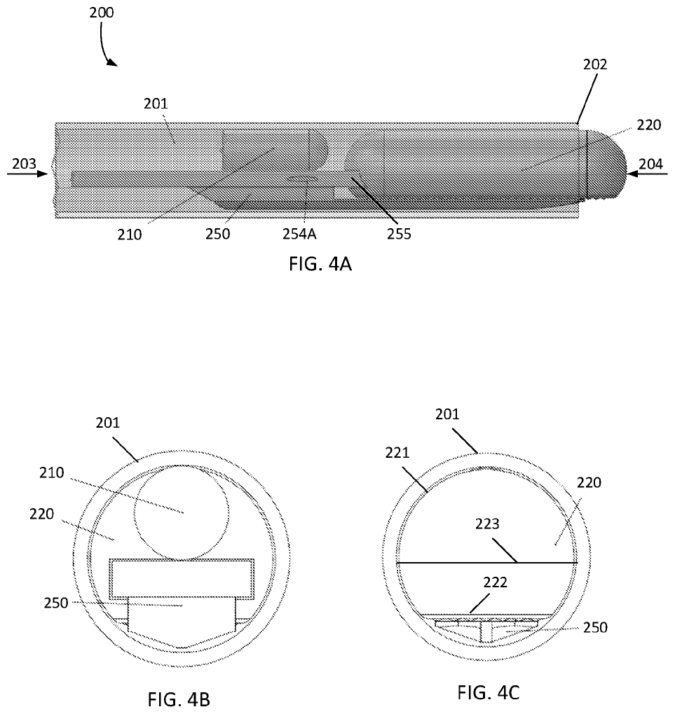

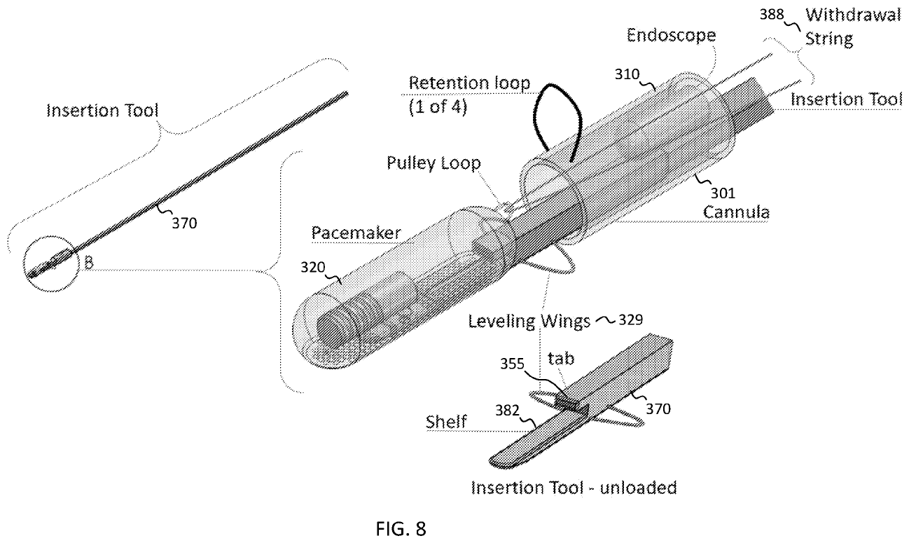

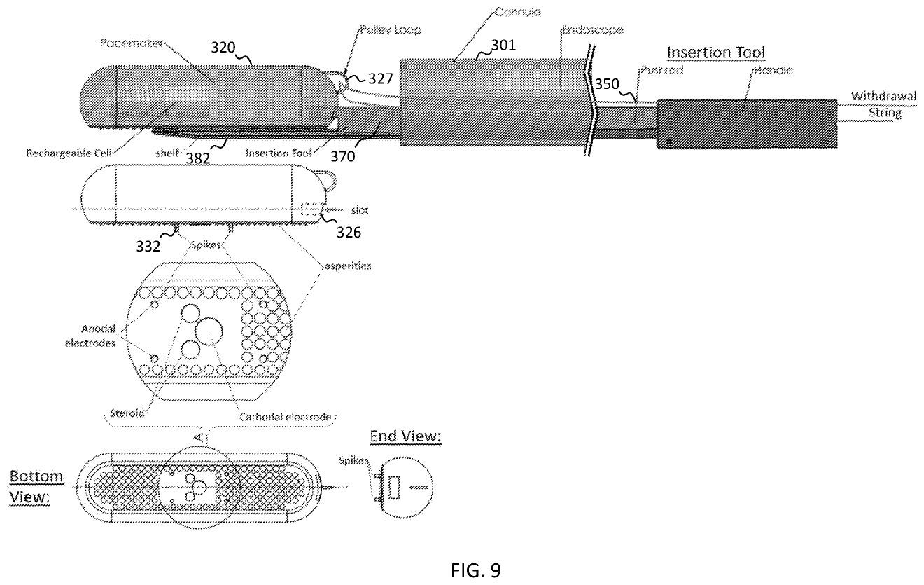

Several different exemplary embodiments are shown and presented in the present subject disclosure. A first exemplary embodiment is shown and described in FIGS. 1-3, a second exemplary embodiment is shown and described in FIGS. 4-7, and a third exemplary embodiment is shown and described in FIGS. 8-9. It should be noted that the features and configurations of each of the exemplary embodiments are not mutually exclusive, in that the features of each embodiment may be substituted into any other embodiment, as would be appreciated by one having ordinary skill in the art. For example, a feature only described in the first exemplary embodiment may be incorporated into the second or third exemplary embodiments, even if not shown and described in the present description and drawings for sake of simplicity.

FIGS. 1A, 1B, and 1C show various views of a pacemaker delivery assembly 100, according to the present subject disclosure. In these and other figures, proximal end 171 refers to the end of the pacemaker insertion tool 170 that is outside the body and controlled by the operator, while distal end 172 refers to the end of the pacemaker insertion tool 170 that is positioned inside the pericardial membrane and delivers the pacemaker 120 into the pericardial space. FIG. 1A shows an overall, oblique view of pacemaker delivery assembly, which has a pacemaker 120 mounted at distal end 172 of an insertion tool 170. Proximal end 171 of insertion tool 170 includes a handle 173 for the operator and other components whose function will be described below, including one or more tubes 176 attached to handle 173, pull rod 177 attached to slide 174 and removable spacer 175 that maintains the distance between handle 173 and slide 174. Handle 173 includes also stanchion 189 and string 188 that passes through tubes 176 and may be tied or otherwise anchored to stanchion 189.

Insertion tool 170 is depicted as straight in FIG. 1 but could be made to be flexible and deflectable for the section in the middle where it consists of only the tubes 176 and pull rod 177. Materials and means for such function are well-known in the art of minimally invasive medical devices.

As depicted in FIG. 1B, the pacemaker delivery assembly 100 includes a cannula 101, which may include a deployable retention feature 105 to keep it from falling out of the pericardium during positioning and release of the pacemaker. An example of retention feature 105 in the form of retractable hoops is shown and presented in Applicant's co-pending application, U.S. patent application Ser. No. 15/552,250, which is incorporated by reference herein in its entirety into this disclosure. In the first part of the implantation procedure, the operator uses standard components and techniques such as are well-known in the art to pass cannula 101 from outside the body so that distal end 102 is within the pericardial space.

An endoscope (not shown) may be included and passed through cannula 101 to visualize potential implanting sites for the pacemaker 120.

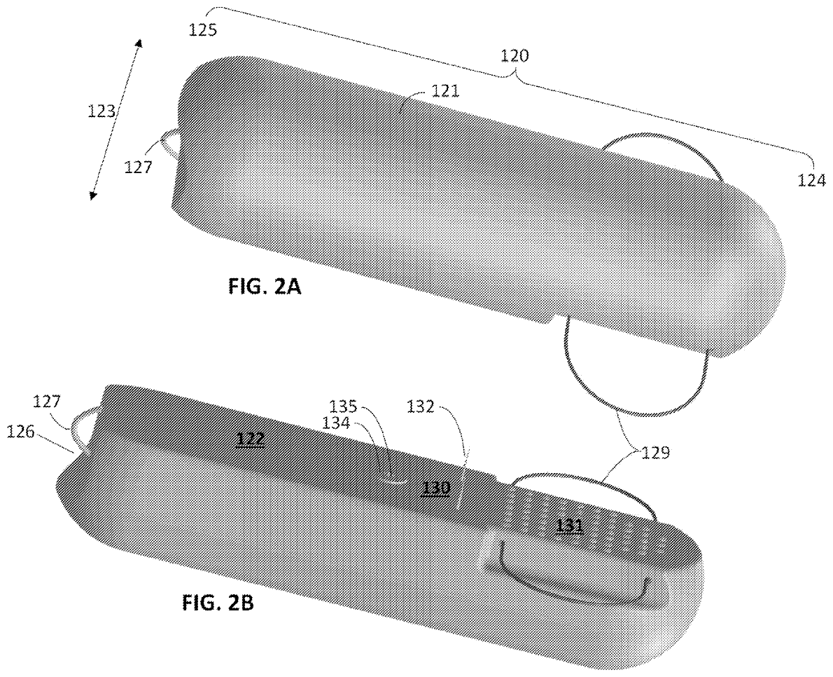

A pacemaker 120 takes up a substantial cross-sectional area of the cannula 101. The pacemaker 120 has a unique shape which will be described herein as “modified cylindrical.” See FIGS. 2A-2B. As used herein and throughout this disclosure, “modified cylindrical” is defined as having an overall cylindrical outer surface 121, with the outer surface 121 having a flattened planar side 122 extending from leading end 124 to trailing end 125 of pacemaker 120, as shown in FIG. 2A. The flattened planar side 122 enables the pacemaker 120 to rest flat and not move or roll over on an epicardial surface. The cross section of the pacemaker 120 must provide sufficient internal volume to the pacemaker to house electronics that power and control pacing function. Further, although shown as substantially round (circular), the cross section of the pacemaker 120 and/or cannula may be other shapes, such as oval.

As shown in FIG. 1B, 1C, 2A and 2B, pacemaker 120 includes wings 129, which further assist in preventing pacemaker 120 from rolling its flattened planar side 122 away from the epicardial surface on which it is implanted. Wings 129 are illustrated as wire loops that project laterally and parallel to flattened planar side 122 near the first end 124. Insertion tool 170 includes a sleeve 186 that projects over pacemaker 120 so that wings 129 are compressed into sleeve 186 so that the pacemaker delivery assembly 100 can be slid freely through cannula 101, as shown in FIG. 1B. When pacemaker 120 is extruded from sleeve 186, wings 129 deploy laterally, as shown in FIG. 1C. Wings may be made of any shape or material to provide this functionality. In one preferred embodiment, the wings are made from nitinol alloy wire approximately 0.15 mm in diameter. Similar wings may also be incorporated onto holder 180 where when deployed they would provide the operator with a sense of the orientation of the epicardial surface. Such wings would fold alongside the holder when it is within cannula 101. One such embodiment is presented below and illustrated in FIG. 8.

FIG. 2A shows a top oblique view of the pacemaker 120, which has a “modified cylinder surface,” or substantially cylindrical outer surface 121, which is advantageously smooth to permit the pericardium to slide over pacemaker 120 as the heart contracts and slides under the pericardium. Wings 129 deploy outward from pacemaker 120 during implantation. The pacemaker 120 has a leading end 124 and a trailing end 125. The leading end 124 and trailing end 125 are substantially similarly shaped as smooth semi-hemispheres, with the trailing end 125 having a recess 126 in which is located a pulley 127.

FIG. 2B shows a bottom oblique view of the pacemaker 120 of FIG. 2A. Additional features visible in this view include pacing electrode 134 and spikes 132 projecting from flattened planar side 122, which includes a smooth surface 130 in the vicinity of pacing electrode 134 and spikes 132 and a textured surface 131 in the vicinity of the leading end 124 and wings 129.

The interior of pacemaker 120 contains the electronic circuitry required to control and power the output pulses that pace the heart (not illustrated). In one embodiment, said electronic circuitry is connected to flat pacing electrode 134 and one or more spikes 132. Advantageously, flat pacing electrode 134 is connected so as to function as a cathode and one or more spikes 132 is connected so as to function as the return electrode(s) or anode. Advantageously, flat pacing electrode 134 is located near the middle of flattened planar side 122, which is made of a dielectric material, so that pacing current passing through pacing electrode 134 is forced through the underlying myocardium to excite it efficiently. Advantageously, the one or more spikes 132 that are connected to function as the return electrode(s) are near and to one side of pacing electrode 134 so as to concentrate pacing current in a small volume of excitable myocardium between them, thereby reducing the amount of electrical power required to capture the myocardium.

FIG. 2B shows a bottom view of the pacemaker 120. In this view, recess 126 and pulley 127 can be visualized. The bottom of the pacemaker 120 has a smooth surface 130 and a textured surface 131. The smooth surface 130 is designed to minimize scar tissue in the vicinity of pacing electrode 134. The textured surface 130 promotes adhesion and may be in the form of bumps spread across a pattern on a portion of the flattened planar side 122. In one embodiment, textured surface 130 is located away from the pacing electrode 134 and spike(s) 132 that are connected as the anode.

The textured surface 131 may be constructed in a variety of ways. For example, shallow asperities may be machined into the mold used to make the device 120. Optionally, the textured surface 130 could also be machined or die-molded into hermetic package made from titanium or green-fired ceramic. Other methods of making such a surface 130 are also possible and within the purview of the present disclosure, as appreciated by one having ordinary skill in the art.

One or more active spikes 132 help anchor the pacemaker 120 in a target location and prevent sliding. Additional spikes that are or are not connected as electrodes may be incorporated to enhance fixation of pacemaker 120 to the location on the epicardial surface where it is originally deployed. The spikes 132 may be in the shape of protruding, sharp metal contacts that act as an effective anode. The pacing electrode 134 and the spikes 132 may be composed of a metal which minimizes impedance, such as, for example, iridium. The spikes 132 may be slender, tapered and highly sharpened to facilitate penetration of the epicardial surface of the heart. The spikes 132 may incorporate barbs along the shaft to increase retention in the myocardium.

As a further means to minimize scar tissue around the electrodes, one or more pieces of drug-eluting material (not illustrated) may be positioned on the underside of the pacemaker 120 in the vicinity of the pacing electrode 134. In the exemplary embodiment illustrated in FIG. 2B, the drug-eluting material resides in a cavity 136 (labeled in FIG. 1C) that lies behind pacing electrode 134. Pacing electrode 134 includes drug-eluting fenestrations 135. Alternatively, drug-eluting material may be coated onto or attached to any surface of pacemaker 120. Drug-eluting materials that inhibit the formation of scar tissue are well-known in the art of implantable devices. They typically include anti-inflammatory agents such as corticosteroids.

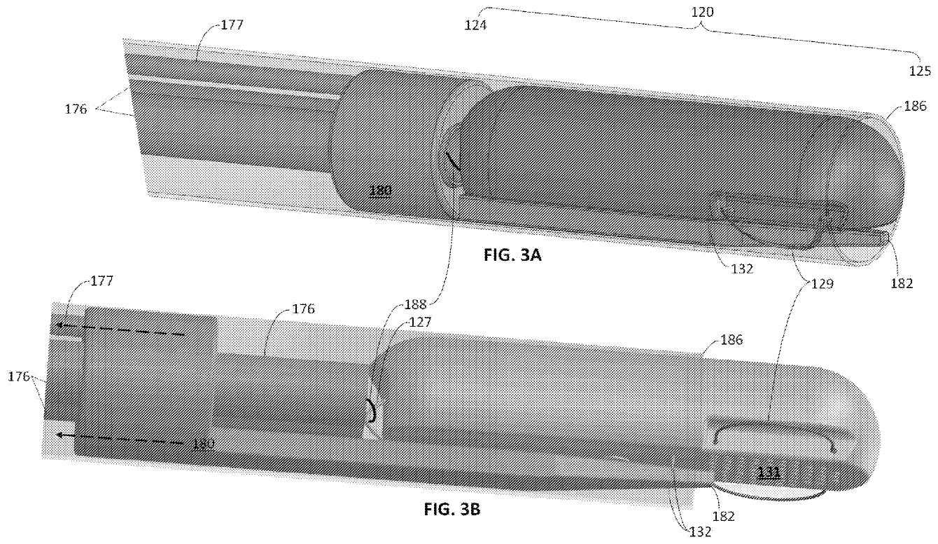

FIG. 3A shows a detailed side view of the distal end 102 of the pacemaker delivery assembly 100. Pacemaker 120 is sitting on shelf 182, which is an extension of holder 180 and which is narrow enough so that spikes 132 can pass to either side of it. Pacemaker 120 is contained within sleeve 186 which holds wings 129 in a furled position and protects sharpened spikes 132 from damage during handling. Pacemaker 120 is pulled against the face of holder 180 by string 188 which passes through tubes 176 and around pulley 127 (better visualized in FIG. 3B). String 188 may be tied or otherwise affixed to stanchion 189 as illustrated in FIG. 1A.

FIG. 3B illustrates the configuration of the distal end 102 of the pacemaker delivery assembly 100 midway through the deployment and release of pacemaker 120. Holder 180 with shelf 182 and attached sleeve 186 is being pulled back by pull rod 177, which is attached to slider 174 at the proximal end of insertion tool 170 as illustrated in FIG. 1A. Tubes 176 are attached to handle 173 at the proximal end of insertion tool 170 as illustrated in FIG. 1A. Spacer 175 is removed by the operator to allow this relative motion. Handle 173 may include a second fixation location for spacer 175 that will lock the insertion tool into the partially retracted configuration illustrated in FIG. 3B. When the pacemaker is in the desired location and orientation on the epicardial surface, string 188 may be untied, cut or otherwise disconnected from handle 173 so that pacemaker 120 may move freely away from the end of insertion tool 170 when spacer 175 is removed and sleeve 186 and shelf 182 are pulled fully back from pacemaker 120 as illustrated in FIG. 1C. Spikes 132 may then engage with and penetrate the epicardial surface. Note that wings 129 have been released and deployed laterally in FIG. 3B and FIG. 1C so that they can prevent axial roll of pacemaker 120, thereby maintaining pacing electrode 134 in contact with the epicardial surface of the myocardium. This configuration is intended to minimize the strength of the pacing pulse required to capture the heart rate, thereby conserving energy from the power supply of pacemaker 120.

When pacemaker 120 is free of the end of insertion tool 170 but still tethered to it by loose thread 188, the operator may send commands to pacemaker 120 to generate electrical output pulses to determine if the pulses capture the heart rate when said pulses are at an acceptable strength. If that is not the case, then thread 188 may be used to pull pacemaker 120 back into sleeve 186 of insertion tool 170 so that it can be redeployed in another location. The close fit between pacemaker 120 and sleeve 186 and shelf 182 permits the pacemaker to return to position on the shelf 182 with the spikes 132 straddling the shelf 182 so that pacemaker 120 can be redeployed or removed without damage. Advantageously, shelf 182 can be made from or incorporate a radio-opaque material to facilitate visualizing its orientation with respect to pacemaker 120 using fluoroscopic imaging.

If the pulses capture the heart rate when their strength is acceptable, pacemaker 120 may be completely released from insertion tool 170 by pulling one end of the thread and dragging it through pulley 127. Stanchion 189 is configured so that the knot or other fixation of thread 188 can be cut away from the end of thread 188 so that it slides freely through tubes 176 and around pulley 127. Insertion tool 170 may then be removed from cannula 101 and retention feature 105 of cannula may be retracted so that cannula 101 may be removed from the patient and the entrance wound from the cannula may be sutured or otherwise closed.

FIGS. 4A, 4B, and 4C show side, back, and front views, respectively, of an alternative embodiment of a pacemaker delivery assembly 200, according to the present subject disclosure. The pacemaker delivery assembly 200 includes a cannula 201, which may include retractable hoops (not shown) at a distal end 202. An example of retractable hoops is shown and presented in Applicant's co-pending application, U.S. patent application Ser. No. 15/552,250, which is incorporated by reference herein in its entirety into this disclosure. When extended, the loops prevent cannula 201 from slipping out of the pericardial sac during pacemaker positioning and release.

An endoscope 210 may be included and used to visualize potential implanting sites for the pacemaker 220. Further, the endoscope 210 may be used to assist in pushing the pacemaker 220 off the end of the pushrod 250.

A pacemaker 220 takes up a substantial volume of the cannula 201. The pacemaker 220 also has a “modified cylindrical” shape which as in the prior embodiment is defined as having an overall cylindrical outer surface 221, with one portion 222 of the outer surface 221 having a flattened planar side, as shown in FIG. 4C. The overall cylinder shape 221 is similar to a standard hot dog or grain silo, for example, with the exception that, in cross section, the flattened (planar) side 222 is viewed as a chord 222. As shown in FIG. 4C, the position of the chord 222 may be variable, as long as the corresponding elements and feature as described in this disclosure allow the pacemaker and its delivery mechanism to function as described herein. The flattened side 222 enables the pacemaker 220 to rest flat and not move or roll over on an epicardial surface. The cross section of the pacemaker 220 needs to provide adequate volume to the pacemaker to enable it to retain its useful features and house electronics, as described in detail below. Further, although shown as substantially round (circular), the cross section of the pacemaker 220 may be other shapes, such as oval, which is generally known as an elliptic cylinder.

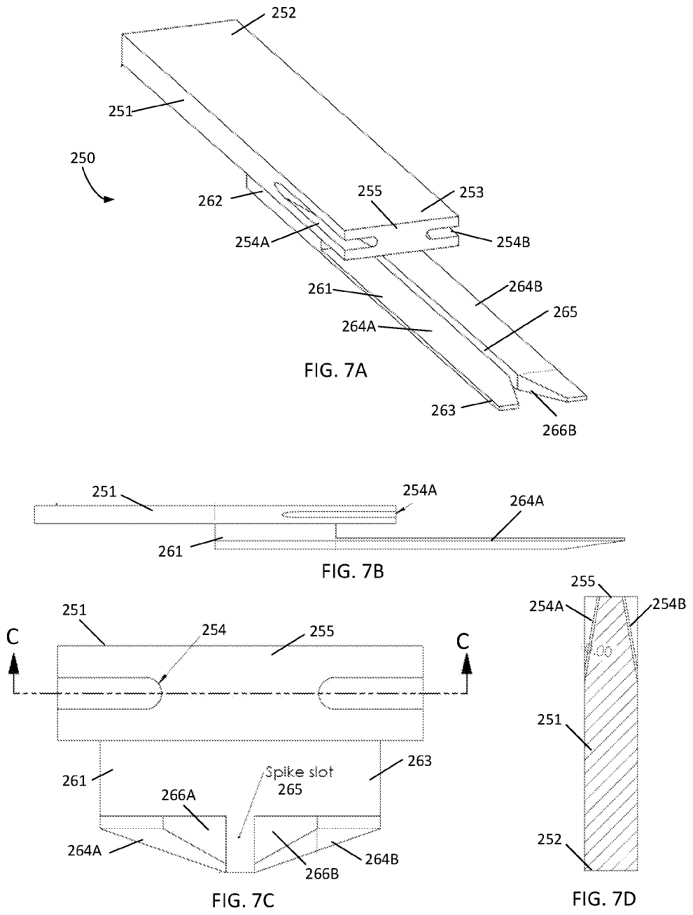

Pushrod 250 has a tab 255 which engages a slot 226 in the back end of the pacemaker 220. Further, as will be presented and described in more detail below, the pushrod 250 includes a shelf underneath the pacemaker 220 to hide the one or more sharp spikes during positioning on an epicardial surface. These features will be described in more detail below with respect to the embodiments shown in FIG. 7.

FIG. 4B shows a back view of the pacemaker delivery assembly 200 from the perspective of the indicator arrow 203 shown in FIG. 4A. From this perspective, the endoscope 210 may be viewed positioned atop the pushrod 250. The pacemaker 220 is evident as it takes up most of the cross section of the interior of the cannula 201.

FIG. 4C shows a front view of the pacemaker delivery assembly 200 from the perspective of the indicator arrow 204 shown in FIG. 4A. From this perspective, the endoscope 210 is not visible at all since the pacemaker 220 blocks it from view. Only the pacemaker 220 and pushrod 250 are visible, as this is the side from which the pacemaker 220 is deployed.

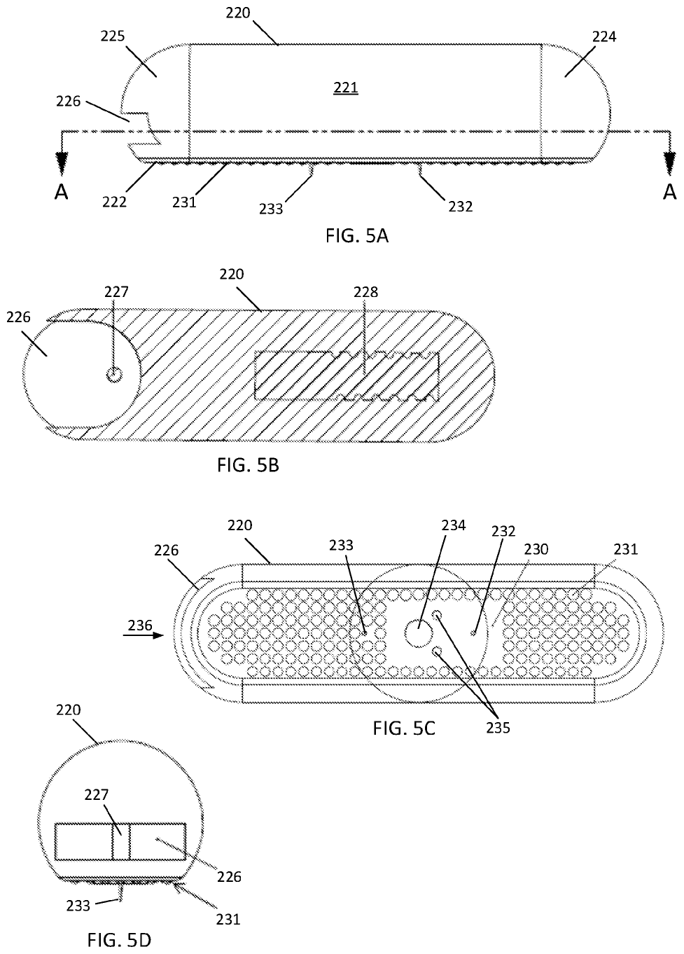

FIG. 5A shows a side view of the pacemaker 220, which has a “modified cylinder surface,” or substantially cylinder outer surface 221 with a flattened side 222. The pacemaker 220 has a leading end 224 and a trailing end 225. The leading end 224 and trailing end 225 are substantially similarly shaped as a semi-hemisphere, with the trailing end 225 having a slot 226 for mating with a tab 255 on the pushrod 250.

FIG. 5B shows a cross-sectional view along plane A-A of the pacemaker 220 of FIG. 2A. The pushrod slot 226 is flat to allow rotation of the engaging pushrod tab 255 to produce similar rotation of the pacemaker 220 during implantation. Pushrod slot 226 further includes a string pole 227, around which a removable string (not shown) may be threaded so that pacemaker 220 can be securely connected to pushrod 250, as will be described in more detail below. The threading of the string to string pole 227 allows the pacemaker 220 to be pulled back into cannula 201 if the target position of the pacemaker 220 is not ideal (e.g., pacing threshold is too high after release from the pushrod 250). String pole 227 may function as a pulley to allow the removable string to be withdrawn, thereby releasing pacemaker 220 freely on the epicardial surface.

An electronic placeholder 228 is cut out within the body of the pacemaker 220 to be able to accommodate electronics needed (not shown) by or associated with the pacemaker 220.

FIG. 5C shows a bottom view of the pacemaker 220. The bottom of the pacemaker 220 has a smooth surface 230 and a textured surface 231. The smooth surface 230 is designed to minimize scar tissue in the vicinity of active electrodes 234. The textured surface 230 promotes adhesion and may be in the form of bumps spread across a pattern at the bottom of the pacemaker 220.

The textured surface 230 may be constructed in a variety of ways. For example, shallow asperities may be machined into the mold used to make the device 220. Optionally, the textured surface 230 could also be machined or die-molded into hermetic package made from titanium or green-fired ceramic. Other methods of making such a surface 230 are also possible and within the purview of the present disclosure, as appreciated by one having ordinary skill in the art.

One or more active spikes 232 help anchor the pacemaker 220 in a target location and prevent sliding. The spikes 232 may be in the shape of protruding, sharp metal contacts that act as an effective anode. The spikes 232 may be composed of a metal which minimizes impedance, such as, for example, iridium.

Further, one or more inactive spikes 233 may also be shaped and function as the active spikes 232 in anchoring the pacemaker 220 into epicardial surface upon implantation, but do not act as anodes.

One active spike 232 and one inactive spike 233 are shown in the figures for sake of simplicity. However, more than one of each type may be positioned on the bottom surface of the pacemaker 220 to provide a stronger grip and anchor into the epicardial surface. Further, the positions of the active spike 232 and inactive spike 233 are centrally located about the centrally positioned electrode 234, but the spikes 232, 233 may also be positioned underneath the ends 224, 225, or other positions or multiple positions, as appreciated by one having ordinary skill in the art.

The electrode 234 is centrally positioned underneath the pacemaker 220. The electrode 234 may comprise of a flat cathode comprised of a metal that minimizes impedance, such as, for example, activated iridium.

Optionally, one or more drug eluting areas 235, in the form of windows or patches, may be positioned on the underside of the pacemaker 220. These areas 235 may be molded, drug eluting polymers exposed on the underneath surface of the pacemaker 120 to inhibit scar tissue formation.

FIG. 5D shows a back view of the pacemaker 220, as it would appear when connecting to the pushrod 250. The string pole 227 is prominent within the pushrod slot 226. Further, the textured surface 231 and the back (inactive) spike 233 are also visible.

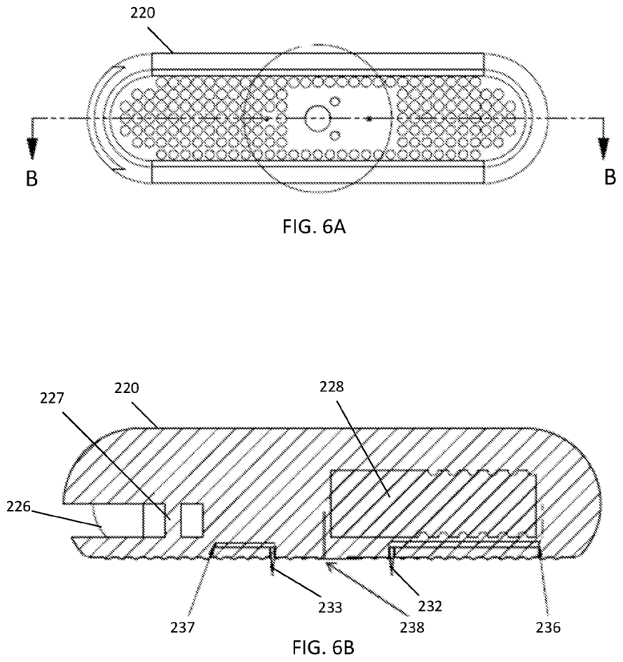

FIG. 6A shows a bottom view of the pacemaker 220, along with a centrally cut plane B-B. FIG. 6B shows a cross-sectional view along plane B-B of the pacemaker of FIG. 6A.

As shown in FIG. 6B, active spike 232 is connected to the electronics (not shown) within the electronic placeholder 228 through an active connection 236. This connection 236 of the anodal spike 232 and a cathode flat electrode 234 to active electronics creates a complete circuit through cardiac tissue to enable pacing.

FIGS. 7A, 7B, 7C, and 7D show various perspectives of pushrod 250. FIG. 7A shows a perspective view of pushrod 250, having a top portion 251, and a bottom portion 261. Top portion 251 acts as a handle and has a back end 252 and a front end 253. The back end 252 can be used to control the movement of the pushrod 250, and ultimately the pacemaker 220. The front end 253 includes a tab 255 that engages with the pushrod slot 226 of the pacemaker 220, as shown in FIG. 7A.

A string slot may be in the form of an aperture (FIG. 7A), or a pair of sloped channels 254A and 254B. As shown in the cross-sectional view along plane C-C of FIG. 7C, FIG. 7D shows that the tab 255 has a pair of sloped channels 254A, 254B to enable a string to be wrapped around the string pole 227, and both ends of the string are able to be pulled back and manipulated by a user. The string may be used to retract the pacemaker 220 to reposition the pacemaker 220 as needed to find the ideal target location on the epicardial surface.

Referring to FIGS. 7A-7C, the bottom portion 261 of the pushrod 250 has a back end 262 and a front end 263. The back end 262 is secured to the bottom of the top portion 251 of the pushrod 250. The front end 263 has an opening to a spike slot 265 that extends a substantial length of the bottom portion 261. The spike slot 265 divides the bottom portion 261 into a pair of tongues 264A, 264B in mirror image. Both tongues 264A, 264B extend a substantial length of the bottom portion 261, and have a mirror image sloped opening 266A, 266B, respectively, which allows a facilitated insertion of the spikes 232, 233 into the spike slot 265. The top surface of the bottom portion 261 of the pushrod 250 accommodates the textured surface 231 at the bottom of the pacemaker 220, while the spikes 232, 233 of the pacemaker 220 slide and rest freely within the spike slot 265, without contact with the surface of the cannula 201 (see FIG. 4C).

Referring to FIGS. 7A-7D, in use, a strand of string (not illustrated) is inserted into one of the string channels 254A or 254B in the top portion 251 of the pushrod 250. Then, the string is pushed through and looped around the string pole 227 in the pushrod slot 226 of the pacemaker 220, and pulled back to insert into the other string channel 254A or 254B in the top portion 251 of the pushrod 250. The pushrod 250 is engaged with a charged and ready pacemaker 220 such that the front end 253 of the top portion 251 of the pushrod 250 is inserted into the pushrod slot 226 of the pacemaker 220 while the spikes 233, and then 232, are inserted into the spike slot 265 in the bottom portion 261 of the pushrod 250. The pacemaker 220 is pushed back on the pushrod 250 so that the spikes 232, 233 are securely within the spike slot 265, the textured surface 231 is positioned on the top surface of the pair of extended tongues 264A, 264B, and the front end 253 of the top portion of the pushrod 250 is securely positioned in the pushrod slot 226 of the pacemaker 220.

Once the pacemaker 220 is securely engaged with the pushrod 250, the combination is inserted into a cannula 201 and lead to a target position within the pericardial sac. When the target position is reached, the pushrod 250 is pushed within the cannula 201 to enable the pacemaker 220 to be deployed into position. The endoscope 210 may also be used to assist in deploying the pacemaker 220.

The spikes 232 and 233 of the pacemaker 220 anchor the pacemaker 220 in position on the epicardial surface while the flattened surface 222 of the pacemaker 220 rests parallel to and layered upon the epicardial surface. The position may be viewed through the endoscope 210.

Further, because of the flattened side 222, a user is able to tactically determine whether the pacemaker 220 has been deployed such that the flattened side 222 is resting flatly on the epicardial surface because of the feedback feel when the pushrod 250 is turned. If the flattened side 222 is resting upon and fully contacting the epicardial surface, then there is resistance to the turning motion of the pushrod 250. If the cylindrical outer surface 221 (and not the flattened surface 222) of the pacemaker 220 is resting upon the epicardial surface, then there is little resistance upon rotation of the pacemaker 220 on the surface. Thus, the user is able to determine whether the desired surface (flattened side 222) is positioned upon the epicardial surface simply by rotating of the pacemaker 220 and evaluating the tactile feedback.

FIGS. 8-9 show another alternative embodiment of the present subject disclosure. Unless specifically described below, the features of the exemplary embodiment presented in FIGS. 8-9 are the same or similar to the features shown and described with respect to FIGS. 4-7 above, and will not be repeated again, for sake of brevity.

The alternative embodiment shown in FIGS. 8-9, and described below, present substitute or additional features, as compared the first embodiment shown and described in FIGS. 1-3, and the second embodiment shown and described in FIGS. 4-7. For example, some additional features include, but are not limited to: (a) Two pairs of spikes 332 on the bottom surface of the pacemaker 320. These pair of spikes 332 straddle the shelf 382 of the insertion tool 370 during introduction of the pacemaker 320. The distal pair is connected to function as the anodal electrode during pacing. (b) The tab 355 on the end of the insertion tool 370 and the slot 326 into which it fits on the pacemaker 320 are relatively smaller than the equivalent features in the second embodiment. (c) There is a pulley loop 327 on the back end of the pacemaker 320 above the slot 326 instead of the channel inside the slot, similar to the first embodiment. The withdrawal string 388 passes through this pulley loop 327 but not through the tab 355 on the insertion tool 370. (d) insertion tool 370 includes leveling wings 329 (similar to wings 129 described previously as a feature of pacemaker 120 in FIG. 2) to enhance the ability of the user to feel the rotational alignment of insertion tool 370 and pacemaker 320 with respect to the epicardial surface.

FIG. 8 shows an oblique (perspective) view of the system as it is deployed in the pericardial sac. At left, the insertion tool 370 is shown its full length, extending beyond the cannula 301 through which it is deployed. Detail B at right shows the relative positions of the insertion tool 370, endoscope 310, and withdrawal string 388 inside the cannula 301, all of which extend outside the patient to the operator (not shown).

FIG. 9 shows various views of the system as it is deployed in the pericardial sac, and external views of the pacemaker 320 by itself. During implantation, the pacemaker 320 sits on the shelf 382 of the insertion tool 370 with the four spikes 332 straddling its shelf 382 and the tab 355 on the insertion tool 370 in the slot 326 at the proximal end of the pacemaker 320. The withdrawal string 388 passes alongside the endoscope 310 and the pushrod 350 of the insertion tool 370 and goes through the pulley loop 327 and back out through the cannula 301. Both ends of the withdrawal string 388 are removably anchored to the handle of the insertion tool 370 such as by tying with a knot, cleating in a slot, or on or under an adhesive strip (not illustrated).

### Procedures for Use

The following description of the procedure of use is more specific to the first embodiment (shown and described in FIGS. 1-3), but in general may be applied to the second embodiment (FIGS. 4-7), and third embodiment (FIGS. 8-9), with minor variations specific to the latter embodiments. Thus, the below procedure will be described without reference numbers but using the terms that are applicable to each of the three described embodiments.

The distal end of the cannula is introduced into the pericardial space under fluoroscopic guidance. This is accomplished according to the conventional clinical procedure of first introducing a wire through a hypodermic needle and then dilating the passageway (often with graded dilators-not illustrated). The cannula may be prevented from accidental dislodgement from within the pericardial sac by deploying retractable retention loops from the distal end of the cannula as illustrated in FIG. 1B. An example of retractable loops is shown and presented in Applicant's co-pending application, U.S. patent application Ser. No. 15/552,250, which is incorporated by reference herein in its entirety into this disclosure.

The insertion tool with the pacemaker loaded onto the shelf and withdrawal string attached through the pulley loop is passed through the cannula and into the pericardial space.

When the pacemaker is in the desired location, the sleeve of the insertion tool may be retracted sufficiently to allow the wings on the pacemaker to deploy. By rotating the insertion tool axially, the operator may feel the wings pushing against the epicardial surface or identify a proper orientation of the wings via fluoroscopy to position the pacemaker so that its flattened surface is parallel to the epicardial surface. The withdrawal string may be loosened from the handle on the insertion tool and the sleeve and shelf may be fully retracted so that the pacemaker is free to engage with the epicardial surface into which the spikes are intended to penetrate. The pacing threshold is determined by introducing stimulus pulses variable amplitude until capture is determined by a physiological monitor such as electrocardiography (ECG).

If the pacing threshold is acceptable, the withdrawal string is pulled out through the pulley loop. It is beneficial to make the withdrawal string from a fine, flexible material that will slide freely past itself and through the tubes and pulley loop. Mercerized cotton-polyester thread is one suitable material, for example. The tubes of the insertion tool provide smooth passages for the thread to prevent the two sides from entangling. The insertion tool may be removed after the thread has been removed from the pulley of the pacemaker. The retention loops on the cannula are retracted and the cannula is removed, leaving the pacemaker in place.

If the pacing threshold is not acceptable, both ends of the withdrawal string are pulled to drag the pacemaker into the sleeve of the insertion tool or into the cannula. The whole pacemaker delivery assembly may then be removed from the cannula and the pacemaker may be refitted to the insertion tool for another attempt at implantation.

### Novel Features

The pacemaker body has a number of advantageous features. The spikes are intended to dig into the surface of the epicardium as the shelf is pulled from under the pacemaker, reducing the possibility that the pacemaker will be dislodged when the withdrawal string is removed. These spikes are advantageously made from a high tensile strength, noble metal such as, for example, pure iridium or various alloys of iridium with platinum. Pure iridium wire stock may be electrolytically etched and the tip sharpened by ion-beam milling, both processes well-known to practitioners of the art. Pure iridium may be electrochemically activated with a conductive surface oxide that provides a low impedance interface for charge injection, as described by (Robblee L S, Lefko J L and Brummer S B (1983) Activated Ir: An electrode suitable for reversible charge injection in saline solution. Journal Electrochemical Society 130: 731-733.). Lowering the impedance in the circuit consisting of the two stimulating electrodes and the intervening tissue reduces the voltage required to push the necessary stimulating current through the tissue, thereby reducing the drain on the power supply.

The arrangement of the electrodes is intended to provide stable, low-threshold pacing capture. Their location on the flat, downward facing, dielectric surface of the pacemaker and their arrangement on that surface forces the applied current of the pacing pulse to stay mostly within the small volume of excitable myocardial tissue immediately beneath and between the electrodes. The distal two spikes may be connected electrically to the pacemaker circuitry to serve as the functional anodal electrode for the pacing pulses (also known as an indifferent or return electrode). A noble metal disk functions as the cathodal electrode for pacing, advantageously protruding slightly proud of the smooth, flat surface of the bottom of the pacemaker that is apposed to the epicardium. Steroid-eluting polymer may be exposed on this bottom surface near the cathodal electrode. Gradual release of an anti-inflammatory steroid such as, for example, dexamethasone reduces the deposition of connective tissue associated with the normal foreign body response of the body to an implanted artificial material. The objective is to keep healthy, excitable myocytes as close as possible to the pacemaker electrodes, thereby reducing the strength of the electrical pulses required to capture the heartbeat. The location of the anodal return electrode(s) as spikes within the myocardium on one side of the cathodal pacing electrode creates a dipole that concentrates the stimulus current through the excitable myocytes.

Reducing the stimulus charge and the required voltage may each or both be used to minimize the electrical energy consumed from the power supply, extending the life of that power supply. Experiments in animals have demonstrated that the pacemaker illustrated in FIG. 2 can capture the ventricular rate using electrical pulses with a charge of 0.05-0.5 microcoulombs and voltages of 0.2-1.5 V. This compares favorably to conventional endovascular pacing, which typically requires a charge of 0.1-1.0 microcoulombs and voltages of 0.5-3.0 V.

Outside the smooth, flat pacemaker surface where the cathodal and anodal electrodes are located, the pacemaker surface may be advantageously roughened by incorporating shallow asperities. This roughened surface is intended to encourage the growth of connective tissue to anchor the pacemaker in place on the epicardial surface. Such asperities may be constructed by molding fine features, indenting, abrading or other methods that are well-known in the art. In addition, such features may include holes/tunnels with a narrower dimension on the surface (and wider dimension internally) to potentially allow fibrous tissues that forms to assist with adhesion of the device to the surface.

The monolithic, leadless design of the present pacemaker eliminates a major source of long-term failure in conventional cardiac pacemakers arising from stress fatigue of flexible components, such as leads and electrodes. In order to fit into the pericardial space and accommodate the pumping motion of the heart, the present pacemaker is advantageously limited in size to a diameter of 4-12 mm and a length of 10-40 mm, depending on the size of the patient. The relatively small internal volume of the present monolithic pacemaker suggests that the amount of energy that can be stored in a primary cell such as lithium may not be sufficient to pace over the remaining life of the patient. Advantageously, the pacing circuitry's power can be provided by a rechargeable cell. Circuitry in the pacemaker to receive externally transmitted power and data such as via an inductive link can be used to recharge the pacemaker whenever indicated.

The construction of the leveling wings on the pacemaker addresses a trade-off of desirable properties. The wings need to be positioned near the bottom, flat surface of the pacemaker and extend some distance laterally to provide a useful feel for and anchoring to the epicardial surface, but they will not fit into the cannula in this orientation. Advantageously they can be made from a highly springy wire so that they will fold up alongside the insertion tool or within the sleeve of the insertion tool as it passes through the cannula and then spring into the desired lateral extension when the leveling wings emerge from the distal end. One suitable material for this wire is nitinol with a diameter of 0.1-0.25 mm. The arched shape of the leveling wings illustrated in FIG. 2 facilitates smooth folding up as they enter the sleeve and cannula and full deployment when they exit.

The insertion tool incorporates various features that facilitate accurate, stable placement of the pacemaker by the operator. It is designed to slip smoothly through the lumen of a cannula that provides percutaneous access to the pericardial space. The pacemaker has a smooth top surface, a flat bottom surface and deployable wings that are designed so as to hold the bottom of the pacemaker flat against the epicardial surface despite the movement of the beating heart within the pericardial membrane. The sleeve at the distal end of the insertion tool holds the pacemaker wings in a folded, retracted position within the sleeve until the sleeve is withdrawn. When the wings are deployed in their lateral orientation, they may provide the operator with haptic feedback about the orientation of the epicardial surface with respect to the axial rotation of the insertion tool and can allow the operator to identify a proper orientation of the wings via fluoroscopy. At the distal end of the insertion tool the pacemaker rests on a shelf that protects the spikes protruding from the bottom, flat surface of the pacemaker until the shelf and sleeve are withdrawn. The insertion tool allows the operator to withdraw the shelf and sleeve while pushing on the trailing end of the pacemaker so as to release the pacemaker onto the surface of the heart in the desired location. Upon such release the spikes can engage with and penetrate the epicardial surface to prevent sliding of the pacemaker. The string allows the pacemaker to float freely on the surface of the heart while the threshold for pacing in that location is measured. The string may then be used to pull the pacemaker back into the insertion tool for repositioning elsewhere on the heart if deemed necessary by the operator. The arrangement of the withdrawal string within the tubes of the insertion tool and through the pulley loop is intended to minimize sliding friction of the string and traction force applied to the pacemaker as the string is withdrawn. Experiments in animals have confirmed that the above steps can be performed reliably and rapidly by an operator using fluoroscopic visualization of the cannula, insertion tool and pacemaker during the implantation process.

The foregoing disclosure of the exemplary embodiments of the present subject disclosure has been presented for purposes of illustration and description. It is not intended to be exhaustive or to limit the subject disclosure to the precise forms disclosed. Many variations and modifications of the embodiments described herein will be apparent to one of ordinary skill in the art in light of the above disclosure. The scope of the subject disclosure is to be defined only by the claims appended hereto, and by their equivalents.

Further, in describing representative embodiments of the present subject disclosure, the specification may have presented the method and/or process of the present subject disclosure as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present subject disclosure should not be limited to the performance of their steps in the order written, and one skilled in the art can readily appreciate that the sequences may be varied and still remain within the spirit and scope of the present subject disclosure.

What is claimed is:

1. A pacemaker device, comprising:
a cylindrical shell having a first end and a second end, and an elongated flattened side which extends from the first end to the second end; and
a flat electrode surface positioned on the flattened side, and functioning as a cathode to the pacemaker device.

2. The pacemaker device of claim 1, further comprising a textured surface on at least some portion of the flattened side to increase adhesion to an epicardial surface.

3. The pacemaker device of claim 1, further comprising one or more elongated spikes protruding from the flattened side, which provide an anti-sliding resistance force between the pacemaker and an epicardial surface.

4. The pacemaker device of claim 3, wherein one or more of the elongated spikes functions as an anode for the pacemaker device.

5. The pacemaker device of claim 4, further comprising a smooth surface on the flattened side in the surface area between the cathode and the one or more anodes.

6. The pacemaker device of claim 1, wherein an anti-inflammatory drug elutes from the flattened side near the cathode.

7. The pacemaker device of claim 6, wherein the anti-inflammatory drug is contained behind the cathode and elutes through perforations in the cathode.

8. The pacemaker device of claim 1, further comprising wings that project laterally and parallel to the flattened side.

9. The pacemaker device of claim 1, further comprising a pulley in one end of the shell to accommodate a removable string whereby the pacemaker device may be withdrawn during an insertion procedure.

10. The pacemaker device of claim 1, further comprising a rechargeable power supply.

11. A pacemaker delivery assembly, comprising:
an elongated cannula;
an insertion tool positionable within the cannula and having a holder portion; and
a pacemaker positionable within the cannula, adapted to engage with the holder portion of the insertion tool, and comprising:
a cylindrical shell having a first end and a second end, and an elongated flattened side which extends from the first end to the second end.

12. The pacemaker delivery assembly of claim 11, wherein the holder portion of the insertion tool has an extended shelf on which the flattened side of the pacemaker may be mounted when engaged with the insertion tool.

13. The pacemaker delivery assembly of claim 11, wherein the pacemaker includes a pulley in one end of the pacemaker to accommodate a removable string whereby the pacemaker may be withdrawn during an insertion procedure.

14. The pacemaker delivery assembly of claim 13, wherein the insertion tool includes a stanchion and at least one tube through which the removable string may pass from the stanchion to the pulley.

15. The pacemaker delivery assembly of claim 11, wherein the insertion tool has a sleeve within which the pacemaker may be positioned when engaged with the holder portion.

16. The pacemaker delivery assembly of claim 11, wherein the pacemaker has wings that project laterally and parallel to the flattened side and which may be folded into the sleeve when the pacemaker is engaged with the holder portion.

17. The pacemaker delivery assembly of claim 11, wherein the pacemaker has one or more elongated spikes protruding from the flattened side, which provide an anti-sliding resistance force between the pacemaker and an epicardial surface.

18. The pacemaker delivery assembly of claim 17, wherein one or more of the elongated spikes functions as an anode for the pacemaker.

19. The pacemaker delivery assembly of claim 11, in which a portion of the insertion tool is flexible and may be deflected as desired.

20. The pacemaker delivery assembly of claim 11, wherein an anti-inflammatory drug elutes from the flattened side near the cathode.

21. The pacemaker delivery assembly of claim 20, wherein the anti-inflammatory drug is contained behind the cathode and elutes through perforations in the cathode.

22. The pacemaker delivery assembly of claim 11, wherein the insertion tool includes a mechanism whereby the operator may pull the shelf and sleeve away from the pacemaker so as to release the pacemaker into the pericardial space.

23. A method of inserting a pacemaker, comprising:
threading a string around a pulley on one end of the pacemaker;
mounting the pacemaker on a first end of an insertion tool;
reversibly attaching both ends of the string to a second end of the insertion tool; and
inserting the insertion tool and pacemaker within a cannula to deliver to a target location on an epicardial surface.

24. The method of inserting a pacemaker of claim 23, further comprising using an insertion tool sleeve to protect an implantation cannula and body tissues from spikes positioned on a flattened side of the pacemaker.

25. The method of inserting a pacemaker of claim 23, wherein the pacemaker has deployable wings.

26. The method of inserting a pacemaker of claim 25, wherein the wings are deployed before releasing the pacemaker in order to determine the correct axial rotation of the insertion tool and pacemaker on the epicardial surface.

27. The method of inserting a pacemaker of claim 23, further comprising loosening the string to detach the pacemaker from the insertion tool and separating the insertion tool from the inserted pacemaker.

28. The method of inserting a pacemaker of claim 27, further comprising withdrawing the pacemaker into the insertion tool by pulling on the string if a pacing function is unsatisfactory.

29. The method of inserting a pacemaker of claim 28, further comprising redeploying the withdrawn pacemaker into a new location on an epicardial surface.

30. The method of inserting a pacemaker of claim 27, further comprising withdrawing the string by pulling on one end, then withdrawing the insertion tool and the cannula and leaving the pacemaker on the epicardial surface.

31. The method of inserting a pacemaker of claim 23, further comprising deflecting the insertion tool to reach the desired location for the pacemaker.
